(19) United States
(12) Patent Application Publication
(10) Pub. No.: US 2009/0163433 A1
(43) Pub. Date: Jun. 25, 2009

(54) ANTHELMINTIC AND/OR INSECTICIDE DEVELOPMENT

(75) Inventors: Warwick Norman Grant, Upper Hutt (NZ); Zainab Ibrahim Issa, Upper Hutt (NZ); Anton Pernthaner, Upper Hutt (NZ); Charles Bix Shoemaker, North Grafton, MA (US); Dairu Shu, Upper Hutt (NZ); Susan Josephine Stasiuk, Palmerston North (NZ)

Correspondence Address: KNOBBE MARTENS OLSON & BEAR LLP, 2040 MAIN STREET, FOURTEENTH FLOOR, IRVINE, CA, 92614, US

(73) Assignee: AgResearch Limited, Hamilton (NZ)

(21) Appl. No.: 12/088,671

(22) PCT Filed: Oct. 2, 2006
(86) PCT No.: PCT/NZ06/00253

§ 371 Date: Sep. 8, 2008

(30) Foreign Application Priority Data
Sep. 30, 2005 (NZ) 539145

(51) Int. Cl. A61K 31/7088; C12N 15/11; C12Q 1/68

(52) U.S. Cl. 514/44; 536/24.3; 435/6

(57) ABSTRACT

The use of a nucleic acid molecule encoding FAS in a nematode or arthropod, or a fragment or variant thereof, to identify or produce FAS as a target for: endectocide; anthelmintic and/or insecticide; development.

### TECHNICAL FIELD

The present invention relates to anthelmintic and insecticide development. In particular, the use of the FAS gene, and/or FAS polypeptide, in nematodes and/or arthropods, and sequence information relating thereto in the development of an anthelmintic and/or insecticide.

### BACKGROUND ART

The treatment of nematode endoparasites and arthropod ectoparasites of livestock (most notably of small ruminants such as sheep) and of companion animals (eg dogs and cats) has been primarily through the use of chemicals targeted to each group of causative organisms: anthelmintics for nematodes and insecticides for arthropods.

More recently, single broad spectrum chemicals known as endectocides have been developed and employed to control both groups of organisms.

The continued use of these compounds has given rise to the development of resistance against them by the target organisms, so that there is a continuing need to develop new classes of compounds for this purpose [1]. The discovery of new classes of compounds is carried out using two approaches. Most currently used chemicals were discovered by large scale random screening of chemicals (usually natural products) against target organisms. More recently, “rational” screens for compounds that affect specific, defined molecular targets have largely superseded random screening. A critical component of these rational screens is the discovery of suitable molecular targets from the causative organism(s).

This invention describes the identification of a molecular target suitable for rational screening for new compounds that will affect nematodes and arthropods and which will have, therefore, utility in the control of endoparasites and ectoparasites of livestock i.e. a new endectocide molecular target. We have demonstrated the validity of this target by showing that inhibition of its function through the technique of RNA interference (RNAi, [2,3]) adversely affects the development and viability of the nematode Trichostrongylus colubriformis (a common endoparasite of small ruminants, especially of sheep and goats) and the sheep body louse, Bovicola ovis, which is an arthropod ectoparasite of sheep.

The target is the multifunctional enzyme, fatty acid synthase or FAS (EC 2.3.1.85). In higher eukaryotes this enzyme catalyses the seven sequential steps in the biosynthesis of long chain fatty acids from acetyl CoA, malonyl CoA and NADPH. It is under consideration as a target for the treatment of obesity [4] and for the treatment of certain tumours [5,6] in humans. Fatty acid biosynthesis is fundamentally different in lower eukaryotes and prokaryotes. In these organisms the single multifunctional enzyme for fatty acid synthesis found in higher eukaryotes is replaced by several enzymes, each of which catalyses one or a small number of steps in the pathway [7]. Several of these are also under consideration as targets for antibacterial drug development [8]. Nematodes and insects posses the multifunctional, higher eukaryote FAS. An important aspect of the novelty of this invention is the demonstration that an invertebrate multifunctional FAS (orthologous to the mammalian enzyme) is essential for development and/or viability. This is clearly not the case in mammals, for example, where compounds which inhibit FAS are not acutely toxic and development of drugs directed at obesity, microbial infection or cancer is therefore feasible. In addition, the lack of acute toxicity associated with FAS inhibition in mammals (the likely host species of parasites against which FAS-targeted endectocides will be employed) implies that such endectocides will be selective for parasites and not toxic to the host.

All references, including any patents or patent applications cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is acknowledged that the term ‘comprise’ may, under varying jurisdictions, be attributed with either an exclusive or an inclusive meaning. For the purpose of this specification, and unless otherwise noted, the term ‘comprise’ shall have an inclusive meaning—i.e. that it will be taken to mean an inclusion of not only the listed components it directly references, but also other non-specified components or elements. This rationale will also be used when the term ‘comprised’ or ‘comprising’ is used in relation to one or more steps in a method or process.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided the use of a nucleic acid molecule encoding FAS in a nematode or arthropod, or a fragment or variant thereof, to identify or produce FAS protein as a target for: endectocide; anthelmintic and/or insecticide; development.

According to a further aspect of the present invention there is provided the use of a nucleic acid molecule encoding FAS in a nematode or arthropod, or a fragment or variant thereof, in the development of: an endectocide; anthelmintic and/or insecticide.

According to another aspect of the present invention there is provided a use of FAS encoded by a gene including a portion thereof corresponding to a nucleotide sequence as set forth in any one of SEQ ID NOs. 1 and 2 or a functional fragment or variant thereof, in the development of: an endectocide; anthelmintic and/or insecticide.

According to another aspect of the present invention there is provided a probe for an FAS gene said probe having a nucleotide sequence corresponding to a sequence as set forth in any one of SEQ ID NOs. 1 or 2 or a functional fragment or variant thereof.

According to an alternative aspect of the present invention there is provided the use of a nematode or arthropod FAS gene or a fragment or a variant thereof as a probe for a FAS gene.

According to a further aspect of the present invention there is provided the use of C. elegans FAS gene or a functional fragment thereof as a probe to a FAS gene.

According to another aspect of the present invention there is provided an isolated nucleic acid molecule having a nucleotide sequence selected from the group consisting of:
a) SEQ ID NOs. 1 and 2;
b) a complement of a sequence in a);
c) a functional fragment or variant of a sequence in a) or b);
d) a homolog or ortholog of a sequence in a), b) or c).

According to another aspect of the present invention there is provided a method of screening for chemical inhibitor compounds of FAS in nematodes or arthropods characterised by the steps of:
a) testing compounds on a test species of nematode or arthropod;
b) determining if a compound inhibits FAS and is therefore a candidate compound.

According to a yet further aspect of the present invention there is provided a further method to determine whether a candidate compound previously identified also inhibits mammalian FAS characterised by the further steps of:
a) substituting a mammalian FAS gene into the test species;
b) testing candidate compounds on the test species;
c) determining if candidate compounds inhibit mammalian FAS.

According to another aspect of the present invention there is provided the use of an RNA sequence which is a complement of a portion of a RNA sequence derived from a nematode or arthropod to disrupt normal FAS activity in a nematode or arthropod. Preferably, the nematode or arthropod may be a parasite.

More preferably, the nematode may be selected from the Family Trichostrongyloideae and/or Trichostrongylus or Caenorhabditis genus.

Most preferably, the nematode may be T. colubriformis or C. elegans, or a species similar thereto.

Preferably, the arthropod may be selected from the Bovicola genus.

Most preferably, the arthropod may be B. ovis, or a species similar thereto. According to a further aspect of the present invention there is provided the use of a nucleic acid encoding mammalian FAS in the development of: an endectocide; anthelmintic and/or insecticide.

Most preferably the mammalian FAS gene may be derived from a rodent, but it may also be derived from an ape or human FAS gene although this list should not be seen as limiting.

The mammalian FAS gene sequence is well known.

For example, the sequence of Rattus norvegicus fatty acid synthase is Genbank Accession Number NM_{—}017332.

According to another aspect of the present invention there is provided a use of FAS encoded by a gene including a portion thereof corresponding to a nucleotide sequence as set forth in any one of SEQ ID NOs. 1 and 2 or a functional fragment or variant thereof, as a target for drug development.

Preferably, the nematode or arthropod may be a parasite.

More preferably, the nematode may be selected from the Family Trichostrongyloideae and/or Trichostrongylus.

Most preferably, the nematode may be T. colubriformis or a species similar thereto.

Preferably, the arthropod may be selected from the Bovicola genus.

Most preferably, the arthropod may be B. ovis or a species similar thereto.

The term ‘test species’ as used herein refers to any arthropod or nematode species in which it is desired to develop a drug which targets the FAS gene of the species which, may or may not be endogenous to the species.

The term ‘candidate compound’ as used herein refers to a compound which inhibits the normal biological function of FAS in a nematode or arthropod to an extent that detrimentally affects development and/or viability of the organism.

The testing may be carried out in a variety of different ways. In general, the testing may be achieved by simply by feeding or otherwise exposing a compound to the test species.

In preferred embodiments in order to identify whether the compound inhibits the FAS enzyme, counter screening may be employed. For example, a counter screen may consist of testing the inhibiting compound against transgenic nematodes which express a mammalian FAS enzyme and demonstration that the mammalian enzyme is not inhibited by the test compound.

Once a candidate compound has been identified it may in some preferred embodiments have its specificity to FAS confirmed by a direct enzyme assay [15, 16].

The FAS gene and FAS polypeptide may be used as a target in rational screening of compounds following principles well known in the art, [13].

The term ‘substituting’ or grammatical variants thereof, as used herein, unless the context provides otherwise, refers to the introduction into the genome of a non-endogenous FAS gene in place of the endogenous FAS gene of a test species which has either been removed or otherwise inactivated.

The substitution of the endogenous FAS nematode gene with a non-endogenous FAS gene, may be generally carried out as follows. First, a mammalian FAS gene may be inserted into the C. elegans genome by any well established method for genetic transformation of C. elegans (which may include but are not limited to the introduction of foreign DNA by microinjection of that DNA into the gonad of adult C. elegans hermaphrodites, [14]. Secondly, the expression of the endogenous gene may be silenced either by the application of RNAi directed specifically against the nematode sequence, or by mutation induced in the endogenous FAS gene to produce a strain of C. elegans in which the endogenous gene is silenced and its function replaced by the mammalian FAS gene.

The term “nucleic acid molecule” as used herein may be an RNA, cRNA, genomic DNA or cDNA molecule, and may be single- or doublestranded. The nucleic acid molecule may also optionally comprise one or more synthetic, non-natural or altered nucleotide bases, or combinations thereof.

The term ‘nucleotide sequence’ as used herein refers to the specific order of nucleotides in a nucleic acid molecule.

The term ‘nucleotide(s)’ as used herein refers to the subunits of DNA (i.e. adenosine (A), guanine (G), thymine (T), or cytosine (C)), and the subunits of RNA (i.e. adenosine (A), guanine (G), uracil (U), or cytosine (C)), which form the basis of the genetic code by the order in which the subunits appear in a DNA or RNA molecule.

By “functional” in relation to nucleic acid molecules and polypeptides it is meant that the fragment or variant is effectively biologically equivalent to full FAS nucleic acid molecule or FAS polypeptide of an arthropod or nematode.

The term ‘variant’ as used herein refers to a nucleic acid molecule or polypeptide wherein the nucleotide or amino acid sequence exhibits substantially 70, 80, 95, or 99% homology with the nucleotide or amino acid sequence as set forth in the sequence listing—as assessed by GAP or BESTFIT (nucleotides and peptides), or BLASTP (peptides), or BLASTX (nucleotides). It should be appreciated that the variant may result from a modification of the native nucleotide or amino acid sequences, or by modifications including insertion, substitution or deletion of one or more nucleotides or amino acids. Where such a variant is desired, the nucleotide sequence of the native DNA may be altered appropriately for example by synthesis of the DNA de novo, or by modification of the native DNA, for example by site-specific or cassette mutagenesis. Preferably, where portions of the cDNA or genomic DNA require sequence modifications, site-specific primer directed mutagenesis is employed using techniques standard in the art. Alternatively, a variant may be naturally occurring. The term variant also encompasses homologous sequences which hybridise under stringent conditions to the sequences of the invention.

The term ‘variant’ also encompasses “conservative substitutions” wherein the alteration of the nucleotide or amino acid sequences, as set out in the sequence listing of this specification, results in the substitution of a functionally similar amino acid residue, [12].

The term ‘fragment nucleic acid molecule’ as used herein refers to a nucleic acid molecule which represents a portion of the nucleic acid molecule of the present invention and is therefore less than full length and comprises at least a minimum sequence capable of hybridising stringently with a nucleic acid molecule of the present invention (or a sequence complementary thereto).

A fragment of a polypeptide of the present invention is a portion of the polypeptide that is less than full length. Preferably the polypeptide fragment has at least approximately 60% identity to a polypeptide of the present invention, more preferably at least approximately an 80% identity, and most preferably at least approximately a 90% identity. Preferably the fragment has size of at least 10 amino acids, more preferably at least 15 amino acids, and most preferably at least 20 amino acids.

Probes are single-stranded nucleic acid molecules with a known nucleotide sequence which is labelled in some way (for example, radioactively, fluorescently or immunologically), which are used to find and mark a target DNA or RNA sequence by hybridizing to it. The creation of probes is well known by those skilled in the art.

The term ‘ortholog’, ‘orthologous gene’, or ‘orthologous polypeptide’ refers to a functionally equivalent yet distinct corresponding nucleotide or amino acid sequence that may be derived from another species. In general an ortholog may have a substantially identical nucleotide or amino acid sequence to the sequences of the present invention as set forth in the sequence listing.

The term ‘anthelmintic’ as used herein refers to a compound having the ability to be harmful to nematodes, and to most preferably, the ability to inhibit growth or development of, or to kill, a nematode.

The term ‘insecticide’ as used herein refers to a compound having the ability to be harmful to nematodes, and to most preferably, the ability to inhibit growth or development of, or to kill, an insect and/or arachnid.

The term ‘endectocide as used herein refers to a compound having the ability to be harmful to arthropods and/or nematodes, and to most preferably, the ability to inhibit growth or development of, or to kill, an insect and/or arachnid.

The term ‘homolog’ refers to a related gene from a different but related species.

The terms complement as used herein are best illustrated by the following example. For the sequence 5′ AGGACC 3′, the complement, is as follows: 3′ TCCTGG 5′.

Thus, preferred embodiments of the present invention may have a number of advantageous utilities which may include:
providing a useful target for development of a new type of: endectodice; anthelmintic or insecticide;
providing a useful target for development of new drugs that target anthelmintic infestations, or arthropod infestations, of humans or mammals;
providing a useful tool for identifying FAS in other nematodes/arthropods;
providing a novel method for screening potential drug candidate compounds.

### BRIEF DESCRIPTION OF SEQUENCE LISTING

SEQ ID NO 1: shows a representative portion of the FAS gene in T. colubriformis
SEQ ID NO 2: shows a representative portion of the FAS gene in B. ovis
SEQ ID NO 3: shows the full FAS gene in C. elegans

### BRIEF DESCRIPTION OF DRAWINGS

Further aspects of the present invention will become apparent from the following description which is given by way of example only and with reference to the accompanying drawings in which:

FIG. 1 delta Ct of B. ovis from dsRNAi feeding assay;

### BEST MODES FOR CARRYING OUT THE INVENTION

### (a) Anthelmintic Development

The validity of FAS as a target for the development of anthelmintic drugs comes from the demonstration that RNA interference (RNAi) mediated knockdown of FAS expression in the free-living (i.e. non-parasitic) nematode Caenorhabditis elegans and in the parasitic nematode Trichostronglyus colubriformis causes death and/or developmental delay in these organisms. The RNAi evidence is presented in Table 1 below.

TABLE 1
Electroporation & feeding nematodes with FAS derived dsRNA

| | | T. colubriformis |
|---|---|---|
| | C. elegans | % iL3 at day 6 |
| FAS dsRNA | Weak effect, some dead | No effect. >80% |
| (feeding with | worms on day 3 | iL3 on day 6 |
| E. coli + CeFAS) | | |
| FAS dsRNA | No effect: all worms were | 8% iL3 on day 6 |
| (feeding with | healthy and started to lay | (92% inhibition |
| E. coli + TcFAS) | eggs on day 4 | of development) |
| FAS dsRNA | Strong developmental | No effect. >80% |
| (electroporation | effect, most adults were | iL3 on day 6 |
| of CeFAS) | sterile with few eggs on | |
| | day 5 | |
| FAS dsRNA | No effect, healthy adults | Strong lethal effect: |
| (electroporation | with lots of eggs | few viable worms at |
| of TcFAS) | | day 6. No iL3 |

Note:
a) phenotype in C. elegans is scored as either viability, or development to fertile adults by day 4 of culture.
b) phenotype in T. colubriformis is scored as viability or the % of larvae that develop to mature infective third stage larvae (iL3) at day 6 of culture

This data shows that:
Delivery to first stage T. colubriformis larvae of dsRNA derived from the T. colubriformis FAS gene results in death and/or severe developmental delay.

This effect is dependent on the sequence of the FAS; delivery to T. colubriformis larvae of FAS dsRNA from C. elegans did not affect viability or development.
The converse is true for delivery of dsRNA to C. elegans larvae. Delivery of C. elegans FAS derived dsRNA inhibits development whereas delivery of T. colubriformis FAS derived dsRNA does not.

The experiments in the table were conducted in two ways. First, newly hatched larvae of T. colubriformis or C. elegans were grown in the presence of Escherichia coli strain HT115 expressing a double-stranded RNA molecule [9, 10] derived from a part of the FAS gene of either the same species (i.e. C. elegans fed with E. coli+ CeFAS) or from the heterologous species (as a negative control for example C. elegans fed with E. coli+TCFAS). The larvae feed on the E. coli and are thus exposed to the dsRNA which is expressed by the bacteria [9, 10]. Second, newly hatched larvae were exposed directly to purified dsRNA from the same segment of the FAS genes as expressed in E. coli. The purified dsRNA was been produced by in vitro transcription (using commercially available reagents kits for this purpose, according to the manufacturer's instructions) then delivered to the nematodes by electroporation. For electroporation, 50-100 newly hatched first stage larvae were suspended in 200 μl trehalose electroporation buffer (272 mM trehalose, 7 mM KH2PO4 (pH6), 1 mM MgSO4) containing 1-2 mg/ml of dsRNA then electroporated at 100V, single pulse in 0.2 cm cuvettes (BioRad GenePulser II). The larvae were recovered by allowing sedimentation under gravity on ice then cultured in liquid NGM with E. coli OP50 as a food source as in 1B. Control worms were electroporated in buffer without RNA then cultured in the same way. For experiments utilising C. elegans as the target species, the phenotype was assessed at day 4 of culture as the proportion of worms remaining alive and/or the proportion of live worms that were fertile adults. The phenotype for T. colubriformis experiments was the proportion of worms remaining alive and/or the proportion of live worms that were mature third stage infective larvae. The data in the table show that for both species, exposure of first stage larvae to dsRNA derived from a part of their own FAS gene results in significant death and/or developmental delay. The extrapolation of these data is that a chemical compound able to inhibit the expression or activity of FAS (i.e. mimic the dsRNA mediated inhibition of FAS activity shown here) would have significant anthelmintic effect.

The segment of the gene from which the dsRNA was derived in these experiments were:

C. elegans: The FAS gene is defined by coordinates 16,759 bp to 25,573 bp of the cosmid F32H2. This sequence was not produced by AgResearch and is in the public domain. Genbank reference Z81523. It is annotated as fatty acid synthase, F32H2.5 in the C. elegans database available at www.wormbase.org.

The DNA sequence of T. colubriformis which is sufficient to (a) induce a FAS specific reduction in FAS mRNA and (b) which has very high homology with other FAS sequences in the public databases, is shown below. Note that this sequence is not in the public domain and was generated by AgResearch. Note also that it is not a complete sequence of the FAS gene but is sufficient to unambiguously identify the sequence as a fragment of the FAS gene.

The portion of the FAS sequence used in these experiments is:

AGANCTAGTGGATCCAACATCTCCAATAGCTCCCCACTGAATGGCGATNC
CCGGATAGCCATCTTCTCGACGTTGCTCGATCATACGTTCCATAGTCGAG
TTCGACCAGCCATAATTGGTCTGACCAGCGTTACCACGTCCCGATGTGAT
CGATGAGAACACGACGAACCACCGAAGAGCCTCGTCAGCAGCTTTGCGGG
TGGCCTGATCGAGATTAATCGTACCGTAGTACTTGGCTTCAGCCGCGTCC
TTGAAATTCTGCACGTTTTGATTTTCGAAAAGACAGTCACGGAGAACCAT
AGCAAGATGGAACACTCCACCTAGACGGCCCATAGAAAGGCACTGGCGGA
TCAGCTCATCAGCATCAGATCTCTTGGCAATATTCAGTGTGGAAATCAGT
ACTGAAATGCCTGTCCTTCTCCAGAAATGCACACATCGTGCCTGATATCC
AGTACGAATACCAGAACGAGATGTGAGCACGAGCTTCCTGGCTCCACGGT
TTATCAGCCATTGGGCAAGTTCGAGTCCAAATCCTCCCAGACCACCAGTG
ATCAGATACACGTGTTGTGGATGGCATAAAGTGCGACAAATTGCACGAAC
AGTGATGTCGGAGGGTAAGCACTTCCGTTGNGGTTCCTNCTTTCGAATTT
CCATCACCACTTTACCCGATATGTTTTCCTGCGGGACATGAACCTGAACG
CCTTTTTAGCCTTGTCAGGNTGGGAACAATGAAGCCGGTACNGGTGCACT
ACACCCTTTTTGATCCAGNCTCAAGTAGCGCGGGTACCTCCTTCCTTNTT
TAAAGTCGNCCACAGTCGG

This sequence has a BLAST score of e⁻¹⁰⁶ with C. elegans F32H2.5: nucleic acid homology of this degree is strong evidence of functional orthology, and the sequence described can be considered to be derived from the T. colubriformis FAS gene.

### (b) Insecticide Development

FAS is also a essential for the viability and/or development of the sheep body louse B. ovis. First instar nymphs of B. ovis were collected from sheep with louse infestation. Sixty nymphs were placed in glass tubes with an artificial louse diet of: naive sheep skin scrapings supplemented at a ratio of 2:1 with E. coli HT115 expressing dsRNA derived from B. ovis FAS. Skin scrapings are collected by scraping the shaved surface of fresh sheep skin. The lice were incubated at 37° C. and 65% humidity for 21 days. The viability and developmental stage of the lice were examined every 7 days and fresh artificial louse diet as described above was added. There were 6 replicate cultures for each dsRNA treatment; two cultures stopped each week and the lice collected for real time PCR analysis of mRNA levels. Control lice (not exposed to dsRNA) reach adulthood under these conditions.

RNAi against FAS led to increased death of nymphs in culture compared to controls and fewer nymphs developed to adulthood. These data are summarised in the Table 2 below. All are significant at p<0.05

TABLE 2
FAS dsRNA in B. ovis

| | Controls | FAS dsRNA |
|---|---|---|
| Dead nymphs (week 1) | 3.3 | 8.8 |
| Dead adults (total) | 5.3 | 11.0 |
| Viable adults (total) | 2.67 | 0.83 |
| Eggs laid. | 46.8 | 32.4 |

These data show clearly that ingestion of dsRNA against FAS results in death or developmental delay in a significant number of lice. We interpret this as evidence that decreased FAS gene expression (as a result of RNAi) or decreased FAS enzyme activity is detrimental to lice and thus that a chemical inhibitor of FAS is likely to be insecticidal.

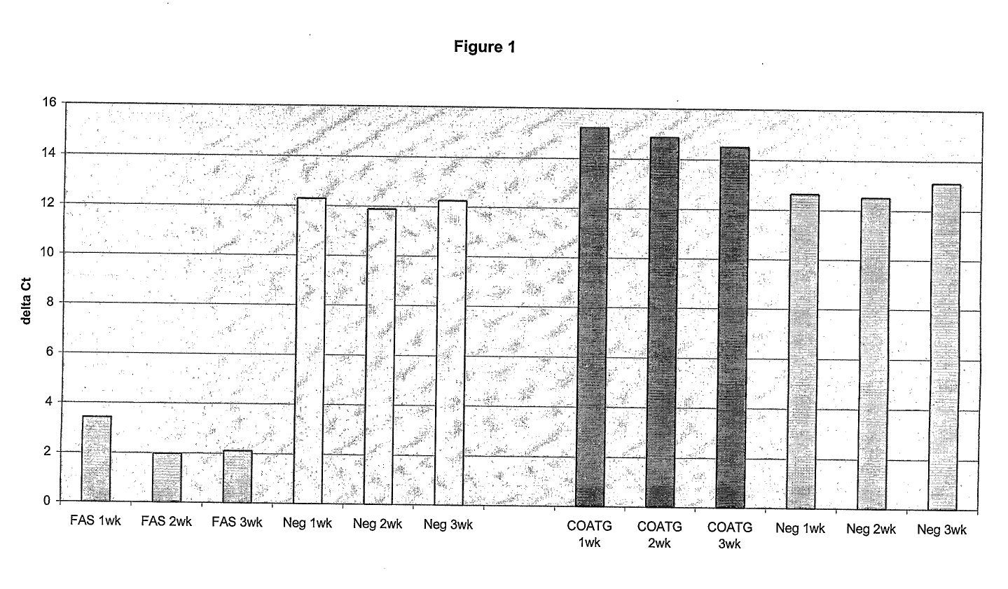

To confirm that exposure of the lice to FAS dsRNA does result in a decrease in FAS gene expression, we collected lice at weekly intervals and measured the relative concentration of FAS mRNA by real time PCR. These data are shown in FIG. 1 which shows the delta Ct of B. ovis dsRNAi feeding assay. Delta Ct is a measure of levels of gene expression. In the context of the FIGURE, delta Ct provides a measure of the degree of inhibition of expression. Large differences in delta Ct are a direct measure of large differences in the level of gene expression, so that if an experimental manipulation results in a decrease in delta Ct, then that manipulation has resulted in a decrease in the level of gene expression. One delta Ct value is equivalent to a two-fold change in expression, so that the scale is a log 2 scale. A change of 1 delta Ct is a 2-fold change, 2 delta Ct is a 4-fold change and so forth, so that a reduction in delta Ct of 4 units is a 16-fold decrease in expression of that gene as can be seen in FIG. 1. The level of FAS mRNA is markedly lower in lice exposed to FAS dsRNA (orange bars) compared to control lice exposed to louse diet supplemented with E. coli HT115 without dsRNA expression (yellow bars). As an additional control, we included lice that were exposed to dsRNA derived from another gene (CoatG) not related to FAS. Exposure to CoatG dsRNA results in a similar degree of lowered viability and developmental arrest as seen with FAS dsRNA.

The DNA sequence of B. ovis which is sufficient to (a) induce a FAS specific reduction in FAS mRNA and (b) which has very high homology with other FAS sequences in the public databases, is shown below. Note that this sequence is not in the public domain and was generated by AgResearch. Note also that it is not a complete sequence of the FAS gene but is sufficient to unambiguously identify the sequence as a fragment of the FAS gene.

ATAATTAGTCCCATAGCAATTCCCGGCAATCAATTCAAACACTATCTTTC
ACTTCGGAAAAATCGGTCAATCATCTGTCTCGTACTTAGTCCCGACCTCT
TCTACAAGTTCCACCAATTAGTAGCTTTAGAAACCCAGGATCCATGGAGG
AGCTGGAGGAGTTGGACAGGCGGCTATTTCCATCGCCTTGTCTATGGGCT
GCAAAGTATTTACTACAGTTGGAACAAAAGAAAAAAGAGAGTTTTTACTA
AAAAGGTTTCCTCAATTAACTGATAACAACATAGGTAATTCGCGTGACAC
TACTTTTGAGCAACATATTCTTCGGCAAACCGGAGGCAAAGGAGTCGACG
TGATTTTAAATTCTTTAGCCGAAGAAAAACTACAAGCGTCCTTGAGGTGT
TTGGGAAAGAATGGAAGATTTTTAGAAATAGGAAAATTTGACCTTTCTAA
TAATACTAAATTAGGAATGGCTATTTTTTTGAAAAATACAGCGTTTCATG
GCATTTTATTAGACAGTTTATTTGATGAATCCGGTCCAGAAAAGTTAGAA
GTTATTAAACTAGTCTCAGAAGGTATTAAATCGGGGGCAGTGAAACCATT
ACCTTTAACTCT

These data show that there is a large and sustained decrease in FAS mRNA following exposure to FAS dsRNA. Exposure to an unrelated dsRNA that induced a similar level of death/developmental delay did not have an effect on FAS dsRNA i.e. that the decrease in FAS mRNA is not a result of decreased louse viability.

### Discussion

Collectively, these data show that FAS is an essential gene for viability and/or development in nematodes and in lice. The hypothesis on which the utility of this invention is based is that a chemical that knocked down FAS activity to the same extent as RNAi would cause a similar degree of developmental delay or death and therefore be potentially useful as an endoectocide in a broad range of nematodes and arthropods. This hypothesis has been tested and validated by others. For example, the enzyme delta-12 desaturase was proposed as a drug target based on RNAi (and other genetic) data and subsequent screening and isolation of chemical inhibitors of this enzyme were shown to be lethal to nematodes [11]. Thus we are confident that the loss of viability (or other phenotypes) induced by exposure to dsRNA are proof that the products of these genes are putative targets for drug development. We note again that FAS inhibition in mammals is not acutely toxic, so that it is likely that chemicals which inhibit nematode or arthropod FAS will not be toxic to mammals even if they inhibit mammalian FAS.

Methods for screening for chemical inhibitors of FAS which are specific for nematodes and/or arthropods (or for invertebrates in general) and do not inhibit mammalian FAS, include:
1. Utilising a screen utilising transgenic C. elegans in which, for example, (a) the C. elegans FAS gene may be inactivated by mutation or knocked down by transgenic RNAi and (b) the mutated/knocked down C. elegans FAS may be replaced by the FAS gene from T. colubriformis or B. ovis. This creates a strain of C. elegans in which viability is dependent on the activity of a parasite derived FAS. An analogous second strain of C. elegans carrying a mammalian FAS gene may also be required. Each of these strains is then tested against a library of candidate chemical compounds. Any compound which is lethal to C. elegans carrying a parasite FAS transgene but not lethal to C. elegans carrying a mammalian FAS transgene is a candidate endectocide.
2. Utilising an in vitro assay able to measure parasite and mammalian FAS activity in a format compatible with high throughput screens, then using this assay to detect chemicals which inhibit parasite FAS activity but do not inhibit mammalian FAS activity.

Aspects of the present invention have been described by way of example only and it should be appreciated that modifications and additions may be made thereto without departing from the scope of the appended claims.

### REFERENCES

1. Sangster, N. C., Anthelmintic resistance: past, present and future. Int J Parasitol, 1999. 29(1): p. 115-24; discussion 137-8.
2. Fire, A., et al., Potent and specific genetic interference by double-stranded RNA in Caenorhabditis elegans. Nature, 1998. 391(6669): p. 806-11.
3. Wang, J. and M. M. Barr, RNA Interference in Caenorhabditis elegans. Methods Enzymol, 2005. 392: p. 36-55.
4. Loftus, T. M., et al., Reduced food intake and body weight in mice treated with fatty acid synthase inhibitors. Science, 2000. 288(5475): p. 2379-81.
5. Kuhajda, F. P., Fatty-acid synthase and human cancer: new perspectives on its role in tumor biology. Nutrition, 2000. 16(3): p. 202-8.
6. Kuhajda, F. P., et al., Fatty acid synthesis: a potential selective target for antineoplastic therapy. Proc Natl Acad Sci USA, 1994. 91(14): p. 6379-83.
7. Schweizer, E. and J. Hofmann, Microbial type I fatty acid synthases (FAS): major players in a network of cellular FAS systems. Microbiol. Mol Biol Rev, 2004. 68(3): p. 501-17, table of contents.
8. Parrish, N. M., et al., Antimycobacterial activity of cerulenin and its effects on lipid biosynthesis. J Antimicrob Chemother, 1999. 43(2): p. 219-26.
9. Timmons, L. and A. Fire, Specific interference by ingested dsRNA. Nature, 1998. 395(6705): p. 854.
10. Timmons, L., D. L. Court, and A. Fire, Ingestion of bacterially expressed dsRNAs can produce specific and potent genetic interference in Caenorhabditis elegans. Gene, 2001. 263(1-2): p. 103-12.
11. McCarter, J. P., Genomic filtering: an approach to discovering novel antiparasitics. Trends Parasitol, 2004. 20(10): p. 462-8.
12. Creighton T. E. ‘Proteins Structure and Molecular Properties.’ WH Freeman and Co. 1984.

13. Young S. Stanley et al Random v Rational Which is better for General Compound Screening, http://www.netsci.org/Science/Screening/feature09.htmi
14. Mello, C. C., Kramer, J. M., Stinchcomb, D., & Ambros, V., (1991) Efficient gene transfer in C. elegans: extrachromosomal maintenance and integration of transforming sequences, The EMBO Journal, 10, 3959-3970
15. Kramer R. & Cohen D. Functional genomics to new drug targets, Nature Reviews Drug Discovery 3, 965-972 (2004)
16. Chanda, S. K. & Caldwell, J. S., Fulfilling the promise: drug discovery in the post-genomic era, Drug Discovery Today; 8(4): 168-74 (2003)

1-21. (canceled)

22. A probe for an FAS gene said probe having a nucleotide sequence corresponding to a sequence as set forth in any one of SEQ ID NOs. 1 or 2 or a functional fragment or variant thereof.

23-24. (canceled)

25. An isolated nucleic acid molecule having a nucleotide sequence selected from the group consisting of:
a) SEQ ID NOs. 1 and 2;
b) a complement of a sequence in a);
c) a functional fragment or variant of a sequence in a) or b);
d) a homolog or ortholog of a sequence in a), b) or c).

26. A method of screening for a chemical inhibitor compound of FAS in nematodes or arthropods characterised by the steps of:
a) testing a compound on a non-embryonic test species of nematode or arthropod; and
b) determining if the compound inhibits FAS and is therefore a candidate compound.

27. A method as claimed in claim 26, further comprising determining whether the candidate compound previously identified also inhibits a mammalian FAS characterised by the further steps of:
a) substituting a mammalian FAS gene into the test species;
b) testing a candidate compound on the test species; and
c) determining if the candidate compound inhibits mammalian FAS.

28-32. (canceled)

33. The method of claim 26, wherein the test species is a nematode.

34. The method of claim 26, wherein the test species is an arthropod.

35. The method of claim 33 wherein the nematode is selected from the family Trichostrongyloideae.

36. The method of claim 33 wherein the nematode is of the genus Trichostrongylus or Caenorhabditis.

37. The method of claim 26 wherein said nematode is selected from the group consisting of:
a) T. colubriformis;
b) C. elegans; and
c) a species similar to a) or b) above.

38. The method of claim 34 wherein the arthropod is selected from the Bovicola genus.

39. The method of claim 38 wherein the arthropod is B. ovis, or a species similar thereto.

40. The method of claim 26, wherein nematode or arthropod expresses an orthologous FAS polypeptide.

41. The method of claim 40, wherein the orthologous FAS polypeptide is derived from a nematode.

42. The method of claim 40, wherein the orthologous polypeptide is derived from an arthropod.

43. The method of claim 27 wherein the mammalian FAS gene is derived from a mammal selected from the group comprising:
a) rodent;
b) ape; or
c) human.

44. A method of disrupting normal FAS activity in a nematode or arthropod comprising:
providing an RNA polynucleotide which is a complement of a portion of an RNA sequence encoding FAS polypeptide derived from a nematode or arthropod; and
administering said RNA polynucleotide in order to contact a nematode or arthropod with said RNA polynucleotide.

45. The method of claim 44 wherein the nematode is selected from the family Trichostrongyloideae.

46. The method of claim 45 wherein the genus is either Trichostrongylus or Caenorhabditis.

47. The method of claim 44 wherein said nematode is selected from the group consisting of:
a) T. colubriformis;
b) C. elegans; and
c) a species similar to a) or b) above.

48. The method of claim 44 wherein the arthropod is selected from the Bovicola genus.

49. The method of claim 48 wherein the arthropod is B. ovis, or a species similar thereto.


SEQUENCE LISTING (3 sequences)

SEQ ID NO: 1 - DNA, 819 bp, Trichostrongylus colubriformis
  misc_feature (4)..(4): n is a, c, g, or t
aganctagtggatccaacatctccaatagctccccactgaatggcgatncccggatagccatcttctcgacgttgctcgatcatacgttccatagtcgagttcgaccagccataattggtctgaccagcgttaccacgtcccgatgtgatcgatgagaacacgacgaaccaccgaagagcctcgtcagcagctttgcgggtggcctgatcgagattaatcgtaccgtagtacttggcttcagccgcgtccttgaaattctgcacgttttgattttcgaaaagacagtcacggagaaccatagcaagatggaacactccacctagacggcccatagaaaggcactggcggatcagctcatcagcatcagatctcttggcaatattcagtgtggaaatcagtactgaaatgcctgtccttctccagaaatgcacacatcgtgcctgatatccagtacgaataccagaacgagatgtgagcacgagcttcctggctccacggtttatcagccattgggcaagttcgagtccaaatcctcccagaccaccagtgatcagatacacgtgttgtggatggcataaagtgcgacaaattgcacgaacagtgatgtcggagggtaagcacttccgttgnggttcctnctttcgaatttccatcaccactttacccgatatgttttcctgcgggacatgaacctgaacgcctttttagccttgtcaggntgggaacaatgaagccggtacnggtgcactacaccctttttgatccagnctcaagtagcgcgggtacctccttccttntttaaagtcgnccacagtcgg

SEQ ID NO: 2 - DNA, 612 bp, Bovicola ovis
ataattagtcccatagcaattcccggcaatcaattcaaacactatctttcacttcggaaaaatcggtcaatcatctgtctcgtacttagtcccgacctcttctacaagttccaccaattagtagctttagaaacccaggatccatggaggagctggaggagttggacaggcggctatttccatcgccttgtctatgggctgcaaagtatttactacagttggaacaaaagaaaaaagagagtttttactaaaaaggtttcctcaattaactgataacaacataggtaattcgcgtgacactacttttgagcaacatattcttcggcaaaccggaggcaaaggagtcgacgtgattttaaattctttagccgaagaaaaactacaagcgtccttgaggtgtttgggaaagaatggaagatttttagaaataggaaaatttgacctttctaataatactaaattaggaatggctatttttttgaaaaatacagcgtttcatggcattttattagacagtttatttgatgaatccggtccagaaaagttagaagttattaaactagtctcagaaggtattaaatcgggggcagtgaaaccattacctttaactct

SEQ ID NO: 3 - DNA, 9014 bp, Caenorhabditis elegans
tattgaagaataatcctgttgatgtgttcaactgttactgaagatgactttccttgcacgaaggtatcgtgatctcctttaactttgagcacctgtaaacaattaattttaagttttgatttagattaaaagaaaagctttaattcactactacaacttgtaaatcttacctcgcaatcttctgacacggccgcaactccataatcttctccaacgtcctcctcacgagcagctccttgctcagctctgatcagtgtgatgtgtccaccgaagtttcttcttggcttgtacctgtaatgtttaaaatttaaaataaattttttaggtttctgaaactcacttgtcagccatgaggaacttggaatgcattgcctcacaggcaaatgcaacggtctctggtgacttgaagagaccagttagcatgacttgatccaccaccttttgcacacgggccttgaatcctggttgttgaaggagttcaaatcggaacttcttgtaatccacattggcgaaacgaagagtcatggcacacatgatctcagattcgaagagtggattgttgaccagggaatctcccgttactccaaaggccatacggtatacctgagaagtttcaaatttaatttcgaaagccataaactagctcaacttacgtttctgtaagtttgcatgtagagatgggatccatcgagaagaatcaatcgttcaacagcatctcttccgtcggactcttgaagcatattggccatttcaaatccaatgcaagctccgtaagaatatccaacaagacgatatggtccagatggttgaatcttcttcatttcacgaatgtagcacttggcaacatcctcgatggaagtttgagggacgttcttggttgattggaagcagtaagctgggaattcacatttgttcataaccttcttgagtggagttgcaattccctcaatcgagtgaacgaagaaagtgattggtccttcttcaaccttgttagccttaacgatgatatcctgtgggtccaaatcattgacatcaactctcatcttaaacaactggttcatttgctgctcaagcatctcagcagtattcaactcggcatcacgttctccatccttcttcatctccaattcgttgacttgaagagcagttctaccagttccaccttgatcagcaagttgttgaagcttgttgagggtcagagtacggatttctttcatggaaagaacaatatcgtgatctctctcaagagcttgcttgatttcgactcccatcaaggaatcaagtcccaaatcactaagattggcatcagcattcaattgagaaatgtcattgactccaagaatatgagcaatagttgccatcaagtctccacctccaacattcttctttgatccgagctcagccttgatgaaggaggaaacaataggatgattccaagacaagaagttgtcaagagaagacaagcaagatggcattctttgtggaagagttccaccaacaacggtgttgttatctcccatgttctcaagaatgacaccgacatctccaatagctccccattgaattgcaattcctgggaatccatcggcacgacgttgatcaatcattctctccatacaagagttggaccatccgtagttcgtttgtccagcatttccacgtccagaagtaatagacgagaagacgacgaaccatttcaagatgttcttgtcacaatgttcacgagaagcataatccaaattaatggttccatagtacttggcctcggcggcatccttgaagttctgcacgttttggttctcgaacagacaatcacgaagaaccatagccaaatggaatattcctccaatcggtcccatggcggtacattggttgatgagttctacagcgtccgatttcttagcaatgttcaaagtggatacaagaacggaaactccagttcttctccagaaatgaacacatcttgcttggtatccagtacgaattccagttctcgaggtaagaacaagtttgcgtgctccacggttgatgagccattgagctaattcaagaccgaatcctccgagtcctccggtgatcaagtacgtgtgttgtggatggcagagagtacgacagatggcacgaactgagatcttggatggtgggcataccttggttccttcatctggacggatctccatgatgacctttccaatgtgttttccagcagacatgaaacggaatgcttcctcagccttgtcagctgggaaggaatgcgagtgaagtggcttgacaactccgctcttgattccttgttccaaaagtctggcaatctctttccattcatccaaatctccaacggttgggtccatgatagaatcaagaaggattccgtgaacggaaacgttatccaaaagcttagccattccaagggatgagttttgagacaagtcgacctttcctatttcaaggaaacgtccgtgacgagccaaacatctgagagaagcttgaagcatctcgttggcaagtgagttgagaacaatgttgactccacgtcctttggtgtgttgacggatatgaagttcgaagtcagcggaacgagagttagcgaagtgatgctcttgaagttgtgggaacaagttcttcaagaattcacgtttctcagctgatccgacagtggtgaagacttcacatccagcagcaagagcgatggcaatggcagcttgtccgactcctccagctcctccgtggatgagaatcttgtctcccttcttcattaatcctcttctaaccaaagcataataagcggtggtgtagacaactgggacagttgaagcttcagcaagagtccaatcccttggaacttcccaagcgtagtctctatcaaccatgacagtggtggcaagagcttgagctggaagaattcccattaaacgggtaccatccttgagacgtccagagaactccattccgagaagacattcacgatcagcaaagtttcctggaatagcatctggtggaagacgtccgtaagcaagcataatatcacggaagttgattggagcataatatacagagcacagttcttgagtagccttgctcttaaccatggaatcaaaatattgatttggagactcgaaccaggtaagagatgaaacatctccacgtgtaagagtgttgatgaaagcgtgctcacaatctttgtagacatgaacatcttcatccttaacaacgatatgacgcattgatccccattgtccatccatgtagtcattggcgtgaagatcaagctcaacgatcttcttggtctcctcatcaccaatattccagactggcggtccatcacgaatttccttgttggcagacatatcaaaagcagaacggaatctgttaatcttcaaattctcctcgacgaagcaaagcccaagtccaactattccattatttcggcatttgttactcactagccagatggtcttggaatctggttcgttgagtctttcctcagagaccttttgaagaggctcaatccagttgaactccttgacatcatcaacgtcaataaagactggatcacgtgggattggtgaaacacgacgaacggcatatgtagtggtcataagggctgggtccgattggaagttgcaaataaggaatccagtggatttgaacatatcgagaagttgttcgtgagtaaagaattgattgtacttacgagaagcatctccaaccatttcatttccaagaagtccttcaatggcaagagcaatttcgtattgagaggtaacttcaacaaccaacaagaatccagtctcgcggatcaaatgcttgcaagcttcaataaaggcaattggatcagccttcttgctgataactttgtccaaaagaacataatccaaattcttgacttctccatgtccagtgaaattattctccaagtcgatcttttgcttctttcctccaataatctcgagcgtactttcgtccaaatgatcaacattggctccgatgcaagtataatcggtttcaagaagtggatgagatgagttggcttcaatgcaatgacgaatttgatcagtagagaccaaatcaaccgctgccattgttgcatgatgcccagcagagttttcaatacaaatatcttgaagactcttgacaatgcgatcattgagaacagctccagcccagagcctatccagttcgaagataccgcgaacacttttcagcttagcagcaacacgatcttcaaagtcctcgcaatcttcaatcttaaacatatcagcgatgtgatggagcacagtgcactttccatcatgagtgaatcttgtgacagtatcatcaagaacaacatcagattgagaagccttgagaacagcaagagcttttacaatttgagctccgttcttcaacttcttcaggagtccagcggattgccatttggagaatccagtgtagagttgttgcttgataacagcagagtagtccttgagaattgtagaagttttcttatgtccactgagacagttatggtcaaagtgaggaacgaaaaagatcttctcatggtagagttgtccagaaacttgaattcttctggcgactgaatgagcgttcaaatcgcagcattcaactcctccagcaatacatccattggtagaatgatcgtttctcaattcaataacttggattccgtctttttcgacaacatgctcaagatgcttgtttggatcaatacgaagatgacgaacgcgagttggaagacgaagagtgtcagatcgctcggcaagaagagcagtttgaagaagggagtccaagaaagtgacccagtttccggtccagtacaagaatccgcgttctccagagttgcatgtcttgtaaattccacggaaagcctgtccatattcatatcctctaagaaggaactccttgtaggcatcctcagtgtccagttcaatacgatcagcaatttcagatgtacgaatatcttccaattttccgtagtagaatggttggttgtcttctggaatgtagattcttccagaagcagcaacttgatcatcagagataatctcaaagtatccatttcctggggacaagacaacatccaatttgactgaaaatattatttaattagaattttaaaagtttaaaataatgtatgaacttacttggcttggtaaggatggtggcagagaagacattgatgttctcaaagacgactggagtcttagtgtaatccaatcccttgagtttgcaaagagttctccaagcaagtaccatatgtccagtgaatgggtagagtacacggccatcaatgacgtgatcaagaagataggtttccttggaatcagttgcaaatggatcaatgttgtaggtagcagatgatgcaacagatccacttcctccagcaagaacttgacgcccatcaatggtaagccagtcttgagtgtggtcccatttccacattggaccaatcattggagttccctttggaacaactcccttgtattgacctcctgggtagagttcagtgatttgaatgtttactcccgcttggtagatctttccaagagatcccaagaagctctcgagttcatcatcattctcagactttggcctgttcataagaccaacgttggtgacagtcttttgaagtgaacgacgaagaatggcttgcatcaatgaatgtggagccatttcaatggtgacagcgtttgctggaatcttttgaatagcttcgtagaacaacactggagagcaagcattgtggacatggtattcagcagagcaagttgcagccaagtcagactcccagtcatcttctgggatggatgtagaaatccactttgaagatcttggttttggttctggaacagcagttctcatgcactcaatcatctcgtcttgaacagctagcattgctggggagtggaatggaattccagatgtatcaacaaccttcgcaaagatatccttctccttgagttgagcacagaatgtagctacaccttcagcatctccagaaatggtaaccgagtcagctccgttgtggcaagcggcaacaactccaggtggagcttgttccttaacctgctcccaagtcaaaccaacagcagccatagcacccttaatttcagtgtgcttcatgatggtggttccacgatgataagcaagtctcattgtttgctcacgagtaattcctccgtcagcatatccacatcccatttctccagttgaatgaccaattattccatctggtgaaactccgagagcagtgagaacatcagtcaaagcgatttggatagcagtgatggcgagcatgcagttcatagtgttgttggagtattgttctggatctggattgcagagcattccgtaaacatcaagtccaaattcttcaagggtcttgctggaagcacgaagagaatcatcgaacattggaatcttcatgagcttgatcgccattccaggccattgggatcccattccagagtaaatgaaatagatcggacgagcttcagtaattgggaccttggtgatgctcttcaaggtctcaacattattttctctatccaaaagcatgtatccacggaatggaagaaggttggctggcatgttagcctgattggccaacaaagcttgaaggtagagatcatccttgttactctcaatttcggtgaaaatgttttcaacagcttcttgagttcttccacagtaactgaaatattttacgttcaaaactgttctgaatctagaaatatatttttaacttacgtgacaagtttggtgaatggatgtggggtgattggtggagctatatgatcagcagccttcaagatgacgtgagtgttagatcctccgaatccgaaagagttgattccaataagtcctcctggaagtgcagttggctccgtgacaaccttcaagcgcccgtcggtaagtcccggaatgtactgatttggagtattgtaatgcaaatttggtggaatcaattgacgttcaatggagagaagaatcttggtcaaagagcaaactccagatgctggctcagcatgtcccatgttagatttgactgaaccgattaaaagagaatcagtacgcttggagcagaaaacttcacagattgcgttggcttcctgtggatctccgaccttagttccagttccgtgagcctcaacgtagtagactgagtttggatcgattccagcctcagaataaacttcttgaagcaattgagcttgtctttctcctgatgggaaagtgattccatgctccttgtgccctaaaatttaaatgtttgagaaaaactcatgttttaattctggaaaaaactcaccatcagtgttagacttggcgtgaacaacagttgcatagagtctttgagccttcttcttgcgttgaatgaagatggctgcaactccttcagtacgacaatatccatcaccagattcgtcgaatgaacggcagcttcccttatctgtgagcatgcccaatctcaagaattggagagctgcagttggagtcaatgtgagatgagctcctgcgacaatagcggcgtcgcattgaccctgaataattaaatattagaataatttttcatctcagattgaaagtagaaatcatcataaattgaacttgcataaaaaacaaagcaacgaacctgtctaatagagtcgacggcaagttgaagtgccaaaagtgaagatgagcaagcggtgtccaccgagaaagatggtccttgcaaatcgaatgtgtaagaaatacggttagagaacatagaacgcacacaacccgtcaacgtgtagcccgttacagtatctgggtcttgtgtgagcattccagaggtttctgaagctgaacatccaacaaaaactcctgttttggatccacggaggtcagtgggatttatgcctgaaatttgagtattattatttaattattccttttttgttttttaattgaataagcactgttcttttcccaatttctttatttgacttgttcaatattccaaaatcataaaagtggaacatcctacttgcacggggtcagtagtgcgcgcgcatgacggtctcaaggttaccaccacagcccggtacatcggggcacactttttccgatttccgtctccttttccgagggagggatatagatagagaatacgagagagaaaaatggacatacccgcatcaaccattgcctcccatgaagcttcaagaagtagtcgaacttgtggatccatgaaatttgcctgttttggagtaacttgaaaaaagcccgcgtcgaatttgttcaacactttcagttttccatgtctttttggcagatcgcaaaatcctggtgtccatctgagagagtcttccgttaccaggtcttcaccagcaagaagcatgtctccgaacattttgacgttatcgcatctgaaaaaattggaacgtttgacatatttcagtaaaatcgtgaacggtttccgcaaaaacttaaaaataagattatttaaaattatttgtaaaattcttaaaaaaagtatattttactttgaaagcttaaatatcggattattttaaacatatcaacggatgatcatttttacagttttgtataggtcaaattaaaatcaaatattagcagtaaaagttgatgaggtgcagttctaatagaggaaacggtaaatgtgatattgtcagtactttcagatgggtttattacattacccaattaccgttctcatccgtcatcaaaattttaggtttcaaaatagatgtaccttggaaatcggcccgagactcctgagattacgatatcgtcctgcttctgccaccattgtgttggatccatgttgtccgaagactgaaattttcaaaattagttacaattttaaaccgtttgaatacaaatctaagaatcgaaatgtgataacaaccatcaaagtggttctatgaaacttattgattttaaaatctcgaaatagaaccagacggacaaaacacaaacacatacgctcacttgtgaatgaggcatgtactcagaaatgacaa